(19) United States
(12) Patent Application Publication
(10) Pub. No.: US 2003/0170698 A1
(43) Pub. Date: Sep. 11, 2003

(54) Droplet-based microfluidic oligonucleotide synthesis engine

(76) Inventors: Peter Gascoyne, Bellaire, TX (US); Jody V. Vykoukal, Houston, TX (US); Jon Schwartz, Sugar Land, TX (US); Frederick F. Becker, Houston, TX (US)

Correspondence Address: Michael C. Barrett, FULBRIGHT & JAWORSKI, L.L.P., Suite 2400, 600 Congress Avenue, Austin, TX, 78701, US

(21) Appl. No.: 10/336,609

(22) Filed: Jan. 3, 2003

Related U.S. Application Data
(60) Provisional application No. 60/345,073, filed on Jan. 4, 2002.

(51) Int. Cl. C12Q 1/68; C07H 21/04; C12M 1/34

(52) U.S. Cl. 435/6; 435/287.2; 536/25.3

(57) ABSTRACT

Devices, systems, and methods for an oligonucleotide synthesis engine. In on embodiment, the invention involves a microfluidic device including: an oligonucleotide synthesis subunit including a reaction surface and means for generating a manipulation force and routing for packet delivery; an analysis or diagnostic subunit; and a control subunit including a program to direct oligonucleotide synthesis. In another embodiment, the invention involves a method for analyzing a sample. An oligonucleotide is synthesized in a solid-phase oligonucleotide synthesis subunit, wherein the subunit includes a reaction surface and means for generating a manipulation force and routing for packet delivery. The synthesis is controlled with a control subunit, wherein the control subunit is programmed for the automatic synthesis of oligonucleotides. The sample is analyzed in an analysis or diagnostic subunit.

This patent application claims priority to, and incorporates by reference, U.S. provisional patent application Serial No. 60/345,073 filed on Jan. 4, 2002 entitled, “Droplet-Based Microfluidic Oligonucleotide Synthesis Engine.”

### BACKGROUND OF THE INVENTION

1. Field of the Invention

The present invention relates generally to fluidic processing and, more particularly, to methods and apparatuses for oligonucleotide synthesis in combination with analysis and/or diagnostics.

2. Description of Related Art

There are many areas in the life sciences such as medicine, pharmacology and pharmacological testing, and environmental detection where the need arises to analyze complex samples which may contain a variety of DNA and RNA target molecules that must be identified and quantified. For example, the need arises in medicine or medical diagnostics to identify the nucleic acids associated with concentrations of proteins, bacteria, viruses, and diseased cells in blood samples.

As the complete human gene has become sequenced, more and more disease states are being associated with particular patterns of gene expression or with the presence of genes containing one or more single nucleotide point mutations (SNPs). Furthermore, more and more viruses and bacteria are being recognized and characterized through their molecular signatures. Commonly, molecular analysis of DNA and RNA is accomplished through the use of molecular probes consisting of oligonucleotide sequences. These are normally synthesized well ahead of the molecular analysis steps, and are commonly purchased from suppliers that specialize in the art of probe synthesis. Clearly, the number of probe sequences needed to identify every pattern of gene expression, every SNP, and every pathogen is astronomically large, making it unfeasible for a single diagnostic device to store presynthesized probes for every molecular sequence of interest.

In light of at least the above, it would be advantageous to provide for technology which allows for the synthesis of probe sequences just prior to the use of the probe sequence. This technology would preferably be able to be programmable for any oligonucleotide sequence.

Shortcomings enumerated in the foregoing are not intended to be exhaustive but rather are among many that tend to impair the effectiveness of previously known processing and fluid injection techniques. Other noteworthy problems may also exist; however, those presented above should be sufficient to demonstrated that apparatus and methods appearing in the art have not been altogether satisfactory and that a need exists for the techniques disclosed herein.

### SUMMARY OF THE INVENTION

In one embodiment, the invention involves a microfluidic device including: an oligonucleotide synthesis subunit including a reaction surface and means for generating a manipulation force and routing for packet delivery; an analysis or diagnostic subunit; and a control subunit including a program to direct oligonucleotide synthesis.

In another embodiment, the invention involves a method for analyzing a sample. An oligonucleotide is synthesized in a solid-phase oligonucleotide synthesis subunit, wherein the subunit includes a reaction surface and means for generating a manipulation force and routing for packet delivery. The synthesis is controlled with a control subunit, wherein the control subunit is programmed for the automatic synthesis of oligonucleotides. The sample is analyzed in an analysis or diagnostic subunit.

In other embodiments, the invention involves the apparatuses, systems, methods, and any applicable software for carrying out aspects of the technology disclosed herein.

### BRIEF DESCRIPTION OF THE DRAWINGS

The following drawings form part of the present specification and are included by way of example and not limitation to further demonstrate certain aspects of the present invention. The invention may be better understood by reference to one or more of these drawings, in which like references indicate similar elements, in combination with the detailed description of specific embodiments presented herein.

FIG. 1 illustrates in block form, an instrument according to one embodiment of the present disclosure containing various subunits, including a subunit that can synthesize oligonucleotide sequences and a separate subunit that can be programmed to direct the sequencing (the control subunit).

### DESCRIPTION OF ILLUSTRATIVE EMBODIMENTS

Within a diagnostic or analytical instrument, a subunit may be created that has the capability to synthesize oligonucleotide sequences under the control of another subunit of the instrument that can be programmed to direct the sequence(s) of the oligonucleotides. The oligonucleotides may then be utilized by the diagnostic or analytical system to accomplish a desired analysis such as, but not limited to, gene discovery, SNP analysis, disease diagnosis, drug discovery, toxicological research, detection of chemical and biological warfare agents, analysis of terrorism agents, pathogen detection, pollution monitoring, water monitoring, fertilizer analysis, food pathogen detection, quality control and blending, massively parallel molecular biological protocols, genetic engineering, oncogene detection, and pharmaceutical development and testing.

Techniques of the present disclosure represent a major improvement over current approaches to oligonucleotide synthesis and analysis using probes. The current approach to molecular analysis is to use oligonucleotide probes that have been manufactured prior to analysis and which are usually held as part of a stock of inventory. As such, prior manufacture demands that every probe needed for a desired analysis is available. In order to comprehensively cover every imaginable molecular sequence that a clinical laboratory might in the future be asked to detect, literally millions of molecular probes would have to be kept readily available as part of an inventory. This obviously would create difficulties associated with storage space, shelf life, ease of locating specific probes, and expense, as well as great potential for human error in providing specified probes to the analysis equipment for a desired analysis.

The ability to synthesize oligonucleotide probes upon demand in a matter of seconds or minutes as taught herein eliminates these complexities and ensures that the correct probe for any desired sequence may be freshly available on demand. Furthermore, the ability to download new sequences from probes to an instrument as taught herein allows for immediate updating of analysis procedures so that they can be kept current with the latest molecular data available on the internet. This allows up-to-the-minute molecular data on new diseases or variances on disease such as influenza, Ebola, etc., to be utilized immediately for diagnostic purposes without the need of having molecular probes manufactured and distributed to analysis points. This allows, for instance, an immediate response to an epidemic for known or new disease. Techniques of this disclosure are also relevant in the detection of biological warfare and bioterrorism agents, where it is anticipated that variances of known biological agents may have been developed as a surprise tactic.

The present disclosure makes it possible to synthesize desired probe sequences where they are required in an analysis apparatus for particular detection or diagnosis problems. This has a huge range of applications ranging from diagnosis in the clinical laboratory, clinic, or even at the point of care, through the research laboratory and environmental protection, including the detection of biological warfare or terrorism. One particular strength of the present invention is its ability to be programmed in real time to direct one or more nucleotide sequences simply by downloading the desired probe sequences and analysis protocol. The need to manufacture and store an inventory of molecular probes is thereby reduced or completely eliminated.

Apparatuses described herein may be readily miniaturized (or made larger) to fit the needs of the user. Its processes may be automated or programmed, manual, or partially automated. The techniques disclosed herein may be used for many different types of microfluidic processing and protocols, and it may be used in processes that are operated in parallel mode, whereby multiple fluidic processing tasks and reactions are performed simultaneously within a single chamber. Areas that may benefit from this technology include, but are not limited to: blood and urine assays, pathogen detection, pollution monitoring, water monitoring, fertilizer analysis, the detection of chemical and biological warfare agents, food pathogen detection, quality control and blending, massively parallel molecular biological protocols, genetic engineering, oncogene detection, and pharmaceutical development and testing.

In one embodiment, the desired configuration can be achieved through the use of a programmable fluidic processor (PFP). The PFP may be utilized to synthesize the oligonucleotide sequences that are downloaded to its control electronics.

I. Dielectrophoretic Fluidic Systems

The use of a PFP (U.S. Pat. No. 6,294,063, which is incorporated herein by reference in its entirety) represents one convenient approach to realizing certain techniques of the current disclosure. A PFP used in conjunction with the teachings herein can accomplish oligonucleotide synthesis, and it is possible to undertake some or all of the analysis steps using these newly synthesized oligonucleotide probes on the same processor, thus blurring the distinction between separate oligonucleotide synthesis and analysis subunits.

U.S. Pat. No. 6,294,063 discloses techniques that relate to the manipulation of a packet of material using a reaction surface, an inlet port, means for generating a programmable manipulation force, a position sensor, and a controller. In one embodiment of that disclosure, material is introduced onto the reaction surface with the inlet port. The material is compartmentalized to form a packet. The position of the packet is tracked with the position sensor. A programmable manipulation force (which, in one embodiment, may involve a dielectrophoretic force) is applied to the packet at a certain position with the means for generating a programmable manipulation force, which may be adjustable according to the position of the packet by the controller. The packet may then be programmably moved according to the programmable manipulation force along arbitrarily chosen paths. One or more packets can be injected onto the reaction surface by methods of the current disclosure instead of using a material inlet port. Similarly, once the packet has entered the reaction surface, reagents and other reacting media can be brought in contact with the packet for a reaction or analysis procedure as taught herein.

The technology described in U.S. patent application Ser. No. ______ entitled “Proofreading, Error Deletion, and Ligation Methods for Synthesis of High-Fidelity Polynucleotide Sequences” filed Jan. 3, 2003 and herein incorporated by reference, can be used in conjunction with techniques of the current disclosure. In one embodiment of that disclosure, an oligonucleotide is made on a reaction surface using a process that finds and eliminates errors in the sequencing. The proofread oligonucleotides are then used without removal from the apparatus.

Other patents and applications that may be used in conjunction with techniques of the current invention include U.S. Pat. No. 5,858,192, entitled “Method and apparatus for manipulation using spiral electrodes,” filed Oct. 18, 1996 and issued Jan. 12, 1999; U.S. Pat. No. 5,888,370 entitled “Method and apparatus for fractionation using generalized dielectrophoresis and field flow fractionation,” filed Feb. 23, 1996 and issued Mar. 30, 1999; U.S. Pat. No. 5,993,630 entitled “Method and apparatus for fractionation using conventional dielectrophoresis and field flow fractionation,” filed Jan. 31, 1996 and issued Nov. 30, 1999; U.S. Pat. No. 5,993,632 entitled “Method and apparatus for fractionation using generalized dielectrophoresis and field flow fractionation,” filed Feb. 1, 1999 and issued Nov. 30, 1999; U.S. patent application Ser. No. 09/395,890 entitled “Method and apparatus for fractionation using generalized dielectrophoresis and field flow fractionation,” filed Sep. 14, 1999; U.S. patent application Ser. No. 09/883,109 entitled “Apparatus and method for fluid injection,” filed Jun. 14, 2001; U.S. patent application Ser. No. 09/882,805 entitled “Method and apparatus for combined magnetophoretic and dielectrophoretic manipulation of analyte mixtures,” filed Jun. 14, 2001; U.S. patent application Ser. No. 09/883,112 entitled “Dielectrically-engineered microparticles,” filed Jun. 14, 2001; U.S. patent application Ser. No. 09/883,110 entitled “Systems and methods for cell subpopulation analysis,” filed Jun. 14, 2001; U.S. patent application Ser. No. 10/005,373 entitled “Particle Impedance Sensor,” by Gascoyne et al., filed Dec. 3, 2001; U.S. patent application Ser. No. 10/028,945 entitled “Dielectric Gate and Methods for Fluid Injection and Control” by Gascoyne et al., filed Dec. 20, 2001; U.S. patent application Ser. No. 10/027,782 entitled “Forming and Modifying Dielectrically-Engineered Microparticles” by Gascoyne et al., filed Dec. 20, 2001; and U.S. patent application Ser. No.______ entitled “Wall-less Channels for Fluidic Routing and Confinement” by Gascoyne et al, filed Jan. 3, 2003; each of which is herein incorporated by reference.

Yet another application that may be used in conjunction with the teachings of the current invention include those described in “Micromachined impedance spectroscopy flow cytometer of cell analysis and particle sizing,” Lab on a Chip, vol. 1, pp. 76-82 (2001), which is incorporated by reference.

Microfluidic processing involves processing as well as monitoring minute quantities of fluid. Processing can be controlled with, for example, a PFP and used in oligonucleotide synthesis, sample analysis, and fluid processing to move a sample and/or oligonucleotide probe from one subunit of a device to a second subunit of the device. Microfluidic processing finds applications in vast fields of study and industry including, for instance, diagnostic medicine, environmental testing, agriculture, chemical and biological warfare detection, space medicine, molecular biology, chemistry, biochemistry, food science, clinical studies, and pharmaceutical pursuits.

a. Programmable Fluidic Processor

A “programmable fluid processor” (PFP) generally includes an electrode array whose individual elements can be addressed with different electrical signals. The addressing of electrode elements with electrical signals may initiate different field distributions and generate dielectrophoretic or other electrical manipulation forces that trap, repel, transport, or perform other manipulations upon packets on and above the electrode plane. By programmably addressing electrode elements within the array with electrical signals, electric field distributions and manipulation forces acting upon packets may be programmable so that packets may be manipulated along arbitrarily chosen or predetermined paths. An impedance sensor or other sensor may also be coupled to an integrated circuit which is coupled to the PFP. The sensor may also be coupled to a controller which is coupled to the PFP. Such a sensor may track the position(s) of packets or particles and feed back the positional information to electrodes so that the correct routing of particles may be ensured.

The electrode array of the PFP may contain individual elements which can be addressed with DC, pulsed, or low frequency AC electrical signals (typically, less than about 10 kHz) electrical signals. One method of switching the voltages to the PFP is a CMOS high voltage chip. Another method uses a discrete switching network for injecting and moving droplets on passivated gold-on-glass PFP arrays.

The PFP is adept at manipulating packets or droplets of sample and reagents and can be used to overcome many difficulties found when using microfluidic valves and other system components. Microfluidic valves tend to be complex and leaky, the mixing of fluids at the ultra-low Reynold's numbers characteristic of small chambers is difficult, microfluidic metering is complicated, and all channel-based designs for these systems have reagent carryover and dead-space issues. Because droplets are discrete and can be efficiently and simply injected with no moving parts under dielectrophoretic control, the quantized metering of samples and reagents may be readily accomplished. Droplets can be moved along arbitrarily chosen and crossing paths by dielectrophoresis on a two dimensional reaction surface, eliminating the need for tubes and the vials required in channel-based fluidic designs. Furthermore, the ability to move droplets along arbitrary, crossing paths allows for full-programmability, and for multiplexed, parallel, and interleaved protocols to be executed.

b. Valves Pumps, Injector, Reagent Metering and Routing

In one embodiment, droplet injection may be a “valving” and “metering” action in which definite volumes of fluid are introduced from a pressurized reservoir (e.g. 2 to 10 psi) by electrically-gated dielectrophoretic forces. The injected droplets carry an intrinsic pressure, stored in the form of surface energy, and this not only induces spontaneous fusion of droplets when they are brought together but also is transferred when a droplet fuses with other fluid allowing, for example, the actuation of fluid flow in a channel. The PFP can be used for switching and metering droplets from several reservoirs and routing them to a reaction accumulator and regions where rinsing is needed. This is an ultra low-power, no moving parts, microscale method to accomplish completely programmable valving, metering and routing, and through the use of pre-pressurized reservoirs, it effectively eliminates the need for pumps.

The programmable fluid processor (PFP) may be configured to act as a programmable manifold that controls the dispensing and routing of all reagents. As used herein, a “program manifold” is meant to describe the combination of computer controlled forces and systems which are used to control the movement of fluids and packets through a biochip. The computer controlled forces are, for example, dielectric forces or magnetic forces. The movements of fluids and packets may be used to: move fluids or packets within a biochip, move fluids or packets into or out of the biochip; initiate or propagate a reaction, separate different components or other function, etc.

Electrode pads can be passivated and coated with anti-wetting agent such as TEFLON so that the routed droplets glide over the reaction surface. In one example, square electrode pads of 30 and 100 μm on a side were used to move droplets from less than one 1 to 6 pad widths; multiple pads can be energized to move larger droplets. The inventors have observed droplets moving at 15 to 4000 μm/sec depending on the dielectrophoresis field. If two droplets are brought together, they will spontaneously fuse making combining their contents easy.

In one embodiment, an injector can be used to inject droplets into the biochip. The static pressure differential necessary to maintain a droplet is expressed by

Pᵢₙ−Pₑₓₜ=γ/r

where Pᵢₙ and Pₑₓₜ are the internal and external hydrostatic pressures, γ the surface tension and r the radius of the droplet. Thus, the pressure differential necessary to maintain a droplet is inversely proportional to the radius of the droplet. Since water adheres to hydrophilic glass, injected droplets tend to remain attached to the tip of the injector pipettes unless the outer surface is made hydrophobic. This can be done by dip-coating the pipettes in an anti-wetting agent such as Sigmacote®, a silicone solution in heptane, or a fluoropolymer, such as PFC1601A from Cytonis, Inc. Similar polar-nonpolar relationships can be used for the solvent systems for oligonucleotide synthesis and determine appropriate injector orifices and field strengths for OSE operations.

c. DEP Forces in Fluidic Systems

For a material of high dielectric constant ε_{d} in a medium of lower dielectric constant εₘ, the time averaged DEP force in response to an alternating, inhomogeneous electrical field E based on the dipole approximation is given by

[symbol]

F_{DEP} [symbol] =2πεₘr³Re

where r is the radius of the material. This force can be used to pull polar liquid droplets into a non-polar suspending phase and to attract droplets to high field regions on a switchable PFP electrode array.

Particles may be fabricated with a dielectric constant that is smaller than the suspending medium at certain frequencies and larger than it at others. Because the magnitude and direction of the DEP force are determined, at least in part, by the relationship between the medium and the particle dielectric constants, εₘ* and ε_{d}* particles may be subjected to attractive or repulsive DEP forces on demand by applying an electric field of appropriate frequency. These principles form one basis for the design of dielectrically engineered beads. Another useful characteristic of dielectrically-engineered beads is that, in an electrical field traveling in the x-direction, they experience a lateral traveling wave dependence of the phase of the field. Within an appropriate band of frequencies, this lateral TWD force can be used to transport a population of beads en masse within a suspending medium, which may form one basis for actuation of metered delivery-on-demand for dielectric beads

Dielectrophoretic forces may be generated by an array of individual driving electrodes fabricated on an upper surface of a reaction surface. The driving electrode elements may be individually addressable with AC or DC electrical signals. Applying an appropriate signal to driving electrode sets up an electrical field that generates a dielectrophoretic force that acts upon a packet contained in an injection tip or vessel. Switching different signals to different electrodes sets up electrical field distributions within a fluidic device. This can be used for the injection of different packets from different injection tips into the device. Such electrical field distributions may be utilized to inject packets into a partitioning medium.

Once the introduction of a first packet has been accomplished, additional packets may be injected through an injector tip or obtained from a region with a different surface energy and fused with the first packet to form a larger packet. In some cases, packet formation at an orifice may proceed until the forming packet becomes detached from the orifice when it touches a previously injected packet. Fluid may be metered out, and packets of different sizes may be made by dielectric injection or by fusing. Since packet injection may occur under the influence of applied electrical fields in one embodiment, automated electrically controlled packet formation may readily be accomplished by switching the fields on and off, or by appropriately adjusting the signals to accomplish the injection of packets. Once injected, packets may be used in situ or else manipulated and moved to desired locations by dielectrophoresis, traveling wave dielectrophoresis, or any other suitable force mechanism following injection.

Although the discussion above relates to dielectrophoretic force(s) aiding in the injection of a fluid packet, it will be understood that any number of different types of forces may be utilized to achieve the fluid packet injection and/or manipulation described herein. Specifically, other separation forces may be employed. For example, acoustic and/or vibrational energy may be used to effectively shake loose a packet from an orifice. If the suspending medium is of low viscosity, such motion-induced packet separation may be inertial. On the other hand, if the suspending medium is of sufficiently high viscosity, then packet detachment may be produced by hydrodynamic drag between the packet and the suspending medium as the orifice is withdrawn sufficiently quickly. With the benefit of the present disclosure, those having skill in the art may choose to rely upon other separation forces. All such other forces sufficient to separate a fluid packet from an orifice onto a surface to achieve metered injection and to manipulate a packet once on a reaction surface fall within the spirit and scope of the present application.

II. Oligonucleotide Synthesis

Based on technology developed for the solid-phase synthesis of polypeptides, the synthesis of nucleic acids with the initial nucleotide attached to certain solid support materials has become possible. The use of a solid support aids in the automation of the synthesis process. Certain syntheses are now routinely carried out using automatic DNA synthesizers by sequentially adding activated monomers to a growing chain that is linked to an insoluble support. (Ike, Y., Ikuta, S., Sato, M., Huang, T., & Itakura, K. (1983) Solid phase synthesis of polynucleotides. Nucl. Acid. Res., 11, 477, herein incorporated by reference).

Synthesis of a specific oligonucleotide sequence may be done using a programmed series of reagent additions to accomplish the extension, washing and deprotection steps as the product is extended. A conventional approach to this problem demands numerous valves and tubes and other fluid handling components that would demand an enormously complex micromechanical system prone to mechanical failure if reduced to chip-scale. The ability to move droplets along arbitrarily chosen and crossing paths on a two dimensional reaction surface eliminates the need for tubes and vials required in microfluidic adaptations of conventional channel-based fluidic designs. The use of a dielectrophoresis (DEP) based programmable fluidic processor (PFP) allows for reconfigurable, channel-less fluid handling, enabling programmed, multiplexed, and/or parallel microfluidic protocols to be executed. This approach reduces or eliminates the need for microfluidic valves, mixers, and explicit metering, and it overcomes carryover and dead-space issues.

An oligonucleotide synthesis system should be able to generate high quality oligonucleotides, use minimal amount of reagents and solvents, and have a very short cycle time for stepwise reactions. The determination of appropriate protocols involve: (a) development of chemistry for derivatization of dielectrically-engineered microbead surfaces with linkers and functional groups suitable for oligonucleotide synthesis; (b) optimization of solvent and reagent systems for oligonucleotide synthesis using DEP-driven delivery; (c) development of methods to monitor oligo synthesis and characterize the final products. One suitable approach is based on the nucleo-phosphoramidite chemistry using trichloroacetic acid (TCA) or other organic acid as the deprotecting agent and, thus far, is one of the most efficient ways to achieve high yield synthesis of oligonucletodies. Other chemistries may also be used, following protocols known in the art.

a. Phosphoramidite Chemistry

Phosphoramidite chemistry can be optimized, for example, by using different solvents for improved dielectrophoretic transport, surface wettability, or volatility. Because reaction mechanisms in oligonucleotide chemistry are well-characterized and studies in developing synthesis protocols using phosphoramidite chemistry, adaptation of reaction parameters, including chemical stochoimetry, reaction times, solution volumes, and solvents are efficient and relatively rapid. Examples of alternative solvent systems include but are not limited to detritylation in propylene carbonate or toluene, and varying the ratio of THF:pyridine in the capping reaction.

Phosphoramidite chemistry involves activation of nucleoside phosphoramidite monomer precursors. The activated monomers are protonated deoxyribonucleoside 3′-phosphoramidites. First, the 3′-phosphorus atom of the phosphoramidite joins to the 5′-oxygen of the growing chan to form a phosphite triester. The 5′-OH of the activated monomer is unreactive because it is blocked by a dimethoxytrityl (DMT) or other protecting group. Coupling is preferably carried out under anhydrous conditions because water reacts with phosphoramidites. In the second step, the phosphite triester is oxidized by iodine to from a phosphotriester (the phosphorus goes from trivalent to pentavalent). Next, the DMT protecting group on the 5′-OH of the growing chain is removed by addition of TCA, dichloroacetic acid, or another organic acid which leaves any other protecting groups intact. The oligonucleotide chain has then been elongated by one base and is ready for another cycle of addition. Specific examples of oligonucleotide synthesis using solution photogenerated acids which are suitable for removal of the acid labile protection group on 5′-O of nucleotides have been described in the art.

The phosphoramidite method, employing nucleotides modified with various protecting groups, is one of the most commonly used methods for the de novo synthesis of polynucleotides. Its reaction efficiency is good for a chemical synthesis scheme and is well suited for the generation of short oligonucleotide probes and primers. The error rate of phosphoramidite oligonucleotide synthesis has been shown to provide a 98.5% stepwise fidelity. This translates to fidelity for a sequence of N bases of (0.985)^{N}.

In one embodiment, a chip-scale implementation of this method using DEP reagent handling on PFP can be used. This stepwise fidelity, however, is highly problematic for synthesizing long polynucleotides because the yield of accurate sequences falls exponentially with sequence length. Living systems contain various enzymatic-proofreading mechanisms for identifying errors in DNA. Several of these have been characterized and adapted for detecting point-mutations in patient samples. These enzymatic methods as well as established chemical cleavage methods may be used in a new modality whereby error-containing polynucleotide sequences are identified, cleaved and eliminated by nuclease digestion, leaving the correctly synthesized sequence intact.

“Error” is defined herein as the error in the stepwise synthesis of a oligonucleotide. Error measurements may involve the percent of the time that a base added to the growing oligonucleotide chain is not the base that was intended to be added to the chain at that position. A synthesis with a high error has a low step-wise fidelity.

b. Solid Support

The use of a solid phase approach is beneficial for oligonucleotide synthesis because the desired product stays on the insoluble support until the final release step. All reactions may occur in a single vessel or on a single chip where excess soluble reagents can be added to drive reactions to completion. At the end of each step, soluble reagents and by-products may be washed away from the beads that bear the growing chains. At the end of the synthesis, NH₃ may be added to remove all protecting groups and release the oligonucleotide from the solid support.

The solid-phase support may be used to retain oligonucleotides after synthesis. Recognizing that the attachment to surfaces of the PFP would compromise the on-the-fly reconfigurability and reusability that is desirable, novel microspheres can be used as mobile solid support for oligo synthesis. The fabrication methods for beads can be modified to provide appropriate microspheres for mixed-solvent systems and develop a traveling-wave DEP delivery-on-demand system for metering, injection and transport of the beads. The ability to reversibly immobilize oligonucleotides in a microfluidic device under electrical control without having to link them directly to the surface of the device represents a major advance in microflume-based molecular analysis and synthesis.

Dielectrically-engineered beads with well-controlled dielectric properties may serve as the solid phase anchors for oligo synthesis. These beads allow attached oligos to be transported and manipulated by traveling-wave DEP or another suitable dielectrically-based force or other force, trapped by positive DEP against fluid flow during rinsing, stirred by alternate DEP trapping and repulsion, and released and flushed from the PFP into receiving stages for further processing after completion of oligonucleotide synthesis. The microspheres can be trapped by positive DEP and repelled by negative DPE by changing the frequency of the applied DEP filed. The microspheres can be fabricated for single and mixed-solvent systems. These microspheres can be metered, infected and transported to the PFP using traveling wave DEP, pressure, and differing surface energies.

Beads can be designed to mimic the dielectric structure of a mammalian cell, and in one embodiment may contain a highly conductive core surrounded by a thin, electrically insulating membrane. The inventors have shown that such microspheres undergo a frequency-dependent change in AC conductivity and can be trapped by positive DEP or repelled by negative DEP by changing the frequency of the applied field. Without being bound by theory, it is believed that this behavior results from a Maxwell-Wagner dielectric dispersion associated with non-conducting shell.

The surface of the beads can be modified to accommodate the chemical requirement of organic synthesis. In a nonlimiting example, the inventors have fabricated engineered microspheres by forming self-assembled insulting monolayers (SAMs) of alkanethiolate and phospholipid on gold-coated polystyrene core particles. Alkanethols CH₃(CH₂)_{η}—SH, chemisorb spontaneously onto gold surfaces to form alkanethiolates that self-organize into densely packed, robust monolayer films. An additional, self-assembled monolayer film of phospholipid can be applied over the alkanethiolate SAM to increase the thickness of the engineered microsphere and yield a polar, hydrophilic outer surface. One bead design that has been shown to be useful consists of gold-coated polystyrene core particles of uniform size (10 microns diameter) that have been coated by with self-assembled monolayers of alkane thiol and subsequently converted to a hybrid bilayer membrane by an additional self-assembled phospholipid monolayer coating step that is able to produce a stable, cross-linked polymeric coat of precisely defined thickness. Engineered microspheres may readily be adapted as a solid phase support for oligonucleotide synthesis.

The effects of spacers, linkage and solid support on the synthesis of oligonucleotides has been described by Katzhendler et al., (1989) while other methods for synthesis on a surface have been described, for example, by LeProust et al. (2000, 2001). The bead design may be adapted for use as oligonucleotide anchors, for example, by the attachment of thiolated oligonucleotide primer sequences or by adding various coatings that allow the attachment of other types of linkers for chemical synthesis, such as polyethyleneglycol terminated with a hydroxyl or silicon based materials.

Thiolated oligonucleotide primers may be attached to engineered microspheres. The ability to reversibly immobilize oligonucleotides in a microfluidic device under electrical control without the use of linkages to the surface of the device represents a major advance in microflume-based molecular analysis and synthesis.

Beads allow the reversible immobilization and transport of oligos under DEP or other electronic control and obviate the need for direct interactions of oligos with the surface of the chip. To allow multiple, sequential syntheses, a bead reservoir and a means to accurately and reliably dispense beads may be used.

The surface of the solid support may include, for example, polystyrene, phospholipid, polyethylene glycol, controlled pore glass or a derivatized membrane. The solid support may include a surface layer which has been designed to bind to the nucleic acid bases for oligonucleotide synthesis and an interior which has been designed to be easily manipulated by external forces. Preferably, the solid support can be manipulated by a dielectric field.

Numerous other materials may be used in the solid support, including but not limited to nitrocellulose, nylon membrane, glass, reinforced nitrocellulose membrane, activated quartz, activated glass, polyvinylidene difluoride (PVDF) membrane, polyacrylamide-based substrate, other polymers such as poly(vinyl chloride), poly(methyl methacrylate), poly(dimethyl siloxane), photopolymers (which contain photoreactive species such as nitrenes, carbenes and ketyl radicals capable of forming covalent links with target molecules and magnetic controlled pore glass described in U.S. Pat. No. 5,601,979, which is incorporated herein by reference.

III. Instrument Synthesis Analysis and Diagnosis Subunits

One embodiment of the present disclosure incorporates two or more subunits of a microfluidic device which can be used together to perform a variety of tasks. These tasks include oligonucleotide synthesis and detection, and may also include proofreading and error deletion of oligonucleotides, analysis of a sample, separation including oligonucleotide and sample purification and sample preparation, sensing position and/or volume of a sample, nucleic acid amplification, nucleic acid hybridization, cell lysis, and a variety of other tasks that are known in the art and can be performed in a chip or microfluidic based system.

One subunit of the device may involve the analysis or diagnosis of a sample. This subunit can, for example, be used to analyze nucleic acids or other biomolecules. Nucleic acid analysis technique may include, for example, nucleic acid hybridization analysis, restriction enzyme analysis, genetic sequence analysis ligase, fluorcytmoetry, and the separation and purification of nucleic acids and proteins. A variety of reporter group such as fluorophores, enzymes and radioisotopes may be used to label the nucleic acid or probe molecule to allow for fluorimetric, calorimetric, or autoradiographic detection. The analysis may also measure the impedance, conductance, electrophoretic movements, or fluorescence of the sample. Similarly, the subunit may be adapted to measure mass ion peaks.

The subunit may also be used to analyze small molecules such as drugs.

A subunit described herein may contain a sensor to sense the position and/or concentration of the sample or other packets on the reaction surface of the subunit. The sensor may be, for example, a flow-through impedance sensor that uses two in-line electrodes driven in counter phase and a common sensor electrode that is used to detect impedance and determine trajectories through the sensor area.

In one aspect of the current disclosure, another subunit may be incorporated in the device to allow for the separation of different components of a sample. This subunit may be fluidically connected to at least one other subunit of the device, and may be controlled by the control subunit. One separation technique that may be used in this subunit is field flow fractionation. Another separation technique involves the use of beads or derivatized beads in a microcolumn (Bruckner-Lea et al., 2000). Electrophoresis and other separation techniques may also be used. This subunit may separate components from the sample before or after the sample is analyzed in the analysis or diagnostic subunit. This subunit is useful, for example, when the sample to be analyzed contains additional elements that could hinder the analysis. For blood samples, it may be preferably to separate components of the blood before analysis.

Another subunit that may be incorporated into the device may lyse cells. This subunit lyses samples with cellular components and releases the crude DNA material along with other cellular constituents. The DNA material may then be separated and analyzed. One method for cell lysis is described by Chandler et al. (2001) where high-frequency ultrasound is focused on a channel of flowing sample.

IV. Control Subunit

A control subunit is a component of one embodiment of the current disclosure which is capable of controlling electrodes and thereby fluid flow within other subunits. The directions used for this control can be downloaded into the control subunit so that oligonucleotides can be synthesized by a synthesis subunit without direct interaction and direction from a user. This can be done by pre-loading the synthesis subunit with, or connecting the synthesis subunit to, the required reagents and substrates for the reaction. For example, the control subunit can be used to automatically direct synthesize an oligonucleotide of up to approximately 100 base pairs.

Sequences that may be useful for synthesis may be downloaded into the control subunit prior to the synthesis of the sequence. In one embodiment, up to 10, 100 or 1000 sequences may be downloaded to the control subunit.

The control subunit may include an electronic device which is connected to one or more other subunits of the instrument.

V. Oligonucleotides

A synthesized oligonucleotide may be subjected to a variety of procedures, including proofreading and error deletion, hybridization, amplification, separation using chromatography or other techniques, and detection using, for example, impedance measurements or analysis using an indicator, mass spectroscopy, or other methods. These procedures can be accomplished while still on the PFP, in a microfluidic subunit attached to the PFP, or after removal from the PFP.

Nucleic acid sequences that are “complementary” are those that are capable of base-pairing according to the standard Watson-Crick complementarily rules. As used herein, the term “complementary sequences” means nucleic acid sequences that are complementary, or capable of hybridizing to each other under stringent conditions such as those described herein. Similarly, the terms “sense” and “antisense” oligonucleotides refers to nucleic acid sequences that are complementary.

a. Nucleic Acid Hybridization

In the present disclosure, a subunit may be used to hybridize oligonucleotides, such as hybridizing sense and antisense oligonucleotides during proofreading and error deletion. As used herein, “hybridization”, “hybridizes” or “capable of hybridizing” shall be understood to mean the forming of a double or triple stranded molecule or a molecule with partial double or triple stranded nature. The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity.

Stringent conditions may include low salt and/or high temperature conditions, such as provided by about 0.02 M to about 0.15 M NaCl at temperatures of about 50° C. to about 70° C. The temperature and ionic strength of a desired stringency may be determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

These ranges, compositions and conditions for hybridization are mentioned by way of non-limiting examples only, and the desired stringency for a particular hybridization reaction may often be determined empirically by comparison to one or more positive or negative controls. Depending on the application envisioned, it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. For example, a medium stringency condition could be provided by about 0.1 to 0.25 M NaCl at temperatures of about 37° C. to about 55° C. Under these conditions, hybridization may occur even though the sequences of probe and target strand are not perfectly complementary, but are mismatched at one or more positions. In another example, a low stringency condition could be provided by about 0.15 M to about 0.9 M salt, at temperatures ranging from about 20° C. to about 55° C. Of course, it is within the skill of one in the art to further modify the low or high stringency conditions to meet a particular application. For example, in other embodiments, hybridization may be achieved under conditions of, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

Nucleic acid segments of the present disclosure may be combined with other DNA sequences to produce a longer segment and may be combined with promoters, enhancers, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and the intended use.

In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

In general, it is envisioned that the hybridization probes known in the art and described herein may be useful as reagents in hybridization. The selected conditions and probes used may depend on the particular circumstances based on the particular criteria required (depending, for example, on the G+C content, type of target nucleic acid, source of nucleic acid, size of hybridization probe, etc.).

b. Nucleic Acid Amplification

Oligonucleotides synthesized with techniques of the present disclosure may undergo amplification, either on the PFP or after removal from the processor. Pairs of primers that selectively hybridize to nucleic acids may be contacted with the isolated nucleic acid under conditions that permit selective hybridization. The term “primer”, as defined herein, encompasses any nucleic acid that is capable of priming the synthesis of a nascent nucleic acid in a template-dependent process. Typically, primers are oligonucleotides from ten to twenty or thirty base pairs in length, but longer sequences can be employed. Primers may be provided in double-stranded or single-stranded form, although the single-stranded form is preferred.

Once hybridized, the nucleic acid primer complex may be contacted with one or more enzymes that facilitate template-dependent nucleic acid synthesis. Multiple rounds of amplification, also referred to as “cycles,” may be conducted until a sufficient amount of amplification product is produced. Next, the amplification product can be detected. In certain applications, detection may be achieved by determining impedance changes. Alternatively, the detection may involve visual detection or indirect identification of the product via chemiluminescence, radioactive scintigraphy of incorporated radiolabel or fluorescent label or even via a system using electrical or thermal impulse signals (Affymax technology).

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, each of which is incorporated herein by reference in its entirety.

Other methods for amplification of the oligonucleotides include: ligase chain reaction (“LCR”), disclosed in EPA No. 320 308, Qbeta Replicase, described in PCT Application No. PCT/US87/00880, Strand Displacement Amplification (SDA), Repair Chain Reaction (RCR), using “modified” primers in a PCR-like, template-and enzyme-dependent synthesis as described in GB Application No. 2 202 328, using an excess of labeled probes where probe binds and is cleaved catalytically as described in PCT Application No. PCT/US89/01025, transcription-based amplification systems (TAS), including nucleic acid sequence based amplification (NASBA) and 3SR (Gingeras et al., PCT Application WO 88/10315), a nucleic acid amplification process involving cyclically synthesizing single-stranded RNA (“ssRNA”), ssDNA, and double-stranded DNA (dsDNA) as described by Davey et al, EPA No. 329 822, a nucleic acid sequence amplification scheme based on the hybridization of a promoter/primer sequence to a target single-stranded DNA (“ssDNA”) followed by transcription of many RNA copies of the sequence as described by Miller et al., PCT Application WO 89/06700, “RACE” and “one-sided PCR” (Frohman, 1990), and methods based on ligation of two (or more) oligonucleotides in the presence of nucleic acid having the sequence of the resulting “di-oligonucleotide”, thereby amplifying the di-oligonucleotide, may also be used in the amplification step of the present invention. Each of the references in this paragraph is incorporated herein by reference.

One example of chip based amplification is, PCT Application No. WO 94/05414, to Northrup and White, incorporated herein by reference, which reports an integrated micro-PCR™ apparatus for collection and amplification of nucleic acids from a specimen.

c. Nucleic Acid Detection

In certain embodiments, it may be advantageous to determine oligonucleotide position and/or hybridization. The oligonucleotide may be detected using impedance measurements. Similarly, a wide variety of appropriate indicator means are known in the art, including fluorescent, radioactive, enzymatic or other ligands, such as avidin/biotin, which are capable of being detected. Fluorescent labels or an enzyme tags such as urease, alkaline phosphatase or peroxidase may be used instead of radioactive or other environmentally undesirable reagents. In the case of enzyme tags, colorimetric indicator substrates are known that can be employed to provide a detection means visible to the human eye or spectrophotometrically, to identify specific hybridization with complementary nucleic acid-containing samples.

In one embodiment, visualization may be used to study the oligonucleotide. A typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation. Visualization may be achieved indirectly. Following separation of amplification products, a labeled, nucleic acid probe may be brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

One example of techniques useful to the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is suited to carrying out aspects of methods according to the present invention. Other examples, U.S. Pat. Nos. 5,304,487 to Wilding et al., and 5,296,375 to Kricka et al., discuss devices for collection and analysis of cell containing samples and are each incorporated herein by reference. U.S. Pat. No. 5,856,174 describes, an apparatus which combines the various processing and analytical operations involved in nucleic acid analysis and is also incorporated herein by reference.

d. Chromatographic Techniques

Separation of proofread oligonucleotides from a reaction mixture can be done by holding the oligonucleotide attached to a solid support by DEP or another force while flowing a solution through the chamber to remove all material that is not bound to the supports (which may include beads). It may also be desirable to separate the oligonucleotides from beads or from other components in a reaction chamber; separation of samples that are obtained to interact with the synthesized oligonucleotides may also be utilized.

Samples may be separated by agarose, agarose-acrylamide or polyacrylamide gel electrophoresis using standard methods. Alternatively, chromatographic techniques may be employed to effect separation. There are many kinds of chromatography which may be used in conjunction with the present disclosure, including but not limited to: adsorption, partition, ion-exchange and molecular sieve, and many specialized techniques for using them including column, paper, thin-layer and gas chromatography. In yet another embodiment, labeled oligonucleotide produces, such as biotin-labeled or antigen-labeled can be captured with beads bearing avidin or antibody, respectively.

Microfluidic techniques include separation on a platform such as microcapillaries, designed by ACLARA BioSciences Inc., or the LabChip™“liquid integrated circuits” made by Caliper Technologies Inc. The automated separation of oligonucleotides in a microfluidic environment has been described by Chandler et al. (2000) and Bruckner-Lea et al. (2000).

e. Mass Spectroscopy

Mass spectrometry provides a means of “weighing” individual molecules by ionizing the molecules in vacuo and making them “fly” by volatilization. Under the influence of combinations of electric and magnetic fields, the ions follow trajectories depending on their individual mass (m) and charge (z). For low molecular weight molecules, mass spectrometry has been part of the routine physical-organic repertoire for analysis and characterization of organic molecules by the determination of the mass of the parent molecular ion. In addition, by arranging collisions of this parent molecular ion with other particles (e.g., argon atoms), the molecular ion is fragmented forming secondary ions by the so-called collision induced dissociation (CID). The fragmentation pattern/pathway very often allows the derivation of detailed structural information. Other applications of mass spectrometric methods known in the art can be found summarized in Methods in Enzymology, Vol. 193: “Mass Spectrometry” (J. A. McCloskey, editor), 1990, Academic Press, New York, which is incorporated by reference.

Due to the apparent analytical advantages of mass spectrometry in providing high detection sensitivity, accuracy of mass measurements, detailed structural information by CID in conjunction with an MS/MS configuration and speed, as well as on-line data transfer to a computer, there has been considerable interest in the use of mass spectrometry for the structural analysis of nucleic acids. Reviews summarizing this field include K. H. Schram (1990); and P. F. Crain (1998). The biggest hurdle to applying mass spectrometry to nucleic acids is the difficulty of volatilizing these very polar biopolymers. Therefore, “sequencing” had been limited to low molecular weight synthetic oligonucleotides by determining the mass of the parent molecular ion and through this, confirming the already known sequence, or alternatively, confirming the known sequence through the generation of secondary ions (fragment ions) via CID in an MS/MS configuration utilizing, in particular, for the ionization and volatilization, the method of fast atomic bombardment (FAB mass spectrometry) or plasma desorption (PD mass spectrometry). As an example, the application of FAB to the analysis of protected dimeric blocks for chemical synthesis of oligodeoxynucleotides has been described (Koster et al. 1987).

Two ionization/desorption techniques are electrospray/ionspray (ES) and matrix-assisted laser desorption/ionization (MALDI). ES mass spectrometry was introduced by Fenn et al. 1984; WO 90/14148 and its applications are summarized in review articles (R. D. Smith et al 1990; B. Ardrey, 1992). As a mass analyzer, a quadrupole is most frequently used. The determination of molecular weights in femtomole amounts of sample is very accurate due to the presence of multiple ion peaks which all could be used for the mass calculation.

MALDI mass spectrometry, in contrast, can be particularly attractive when a time-of-flight (TOF) configuration is used as a mass analyzer. The MALDI-TOF mass spectrometry has been introduced by Hillenkamp et al. (1990). Since, in most cases, no multiple molecular ion peaks are produced with this technique, the mass spectra, in principle, look simpler compared to ES mass spectrometry. DNA molecules up to a molecular weight of 410,000 Daltons could be desorbed and volatilized (Williams et al, 1989). More recently, this the use of infra red lasers (IR) in this technique (as opposed to UV-lasers) has been shown to provide mass spectra of larger nucleic acids such as, synthetic DNA, restriction enzyme fragments of plasmid DNA, and RNA transcripts up to a size of 2180 nucleotides (Berkenkamp et al., 1998). Berkenkamp et al., 1998, also describe how DNA and RNA samples can be analyzed by limited sample purification using MALDI-TOF IR.

In Japanese Patent 59-31909, an instrument is described which detects nucleic acid fragments separated either by electrophoresis, liquid chromatography or high speed gel filtration. Mass spectrometric detection is achieved by incorporating into the nucleic acids atoms which normally do not occur in DNA such as S, Br, I or Ag, Au, Pt, Os, Hg.

VI. Definitions

As used herein, a “carrier fluid” refers to matter that may be adapted to suspend other matter to form packets on a reaction surface. A carrier fluid may act by utilizing differences in hydrophobicity between a fluid and a packet. For instance, hydrocarbon molecules may serve as a carrier fluid for packets of aqueous solution because molecules of an aqueous solution introduced into a suspending hydrocarbon fluid will strongly tend to stay associated with one another. This phenomenon is referred to as a hydrophobic effect, and it allows for compartmentalization and easy transport of packets. A carrier fluid may also be a dielectric carrier liquid which is immiscible with sample solutions. Other suitable carrier fluid include, but are not limited to, air, aqueous solutions, organic solvents, oils, and hydrocarbons.

As used herein, a “partitioning fluid” refers to any matter that may be adapted to suspend and compartmentalize other matter to form packets on a reaction surface or a veil between two fluids. A partitioning fluid medium may act by utilizing differences in hydrophobicity between a fluid and a packet. For instance, hydrocarbon molecules may serve as a partitioning medium for packets of aqueous solution because molecules of an aqueous solution introduced into a suspending hydrocarbon fluid will strongly tend to stay associated with one another. This phenomenon is referred to as a hydrophobic effect, and it allows for compartmentalization and easy transport of packets upon or over a surface. A partitioning fluid may also be a dielectric carrier liquid which is immiscible with sample solutions. Other suitable partitioning fluids include, but are not limited to, air, aqueous solutions, organic solvents, oils, and hydrocarbons.

As used herein, an “immiscible fluid” refers to any matter that does not mix with the surrounding fluid, and can be used as a partitioning fluid. For example, the immiscible fluid may be an aqueous solution surrounded by a hydrocarbon partitioning medium.

As used herein, a “programmable fluid processor” (PFP) refers to a device that may include an electrode array whose individual elements can be addressed with different electrical signals. The programmable fluid processor (PFP) can be configured to act as a programmable manifold that controls the dispensing and routing of reagents. As used herein, a “program manifold” is meant to describe the combination of computer controlled forces and systems which are used to control the movement of fluids and packets through a biochip. The computer controlled forces are, for example, dielectric forces, magnetic forces, or any other electrically, mechanically, or optically based forces. The movements of fluids and packets may be used to: manipulate fluids or packets within a biochip, move fluids or packets into or out of the biochip; initiate or propagate a reaction, separate different components or other function, etc.

As used herein, a “biochip” refers to a biological microchip which can be described as a nucleic acid biochip, a protein biochip, a lab chip, or a combination of these chips. The nucleic acid and protein biochips have biological material such as DNA, RNA or other proteins attached to the device surface which is usually glass, plastic or silicon. These biochips are commonly used to identify which genes in a cell are active at any given time and how they respond to changes. The lab chip uses microfluidics to do laboratory tests and procedures on a micro scale. A design of a biochip that is a PFP-based general-purpose bioanalysis apparatus may be termed a “BioFlip.”

A used herein, a “reaction surface” is a surface or a volume which provides an interaction site for packets. In one embodiment, the reaction surface may be completely or partially covered with a partitioning medium or other substance. The reaction surface may be defined by walls or by a change in the surface energies of the top or bottom surfaces surrounding the reaction surface. For example, the reaction surface may be coated with a hydrophilic material while the surrounding surfaces have a hydrophobic surface.

As used herein, an oligonucleotide synthesis engine (OSE) is a microfluidic device that may exploit a wide range of effects that become dominant on the microfluidic scale including but not limited to the hold-off properties of capillary tubes; the high pressures intrinsic to tiny droplets; the tendency of droplets to fuse and rapidly mix on contact with miscible solvents; the attractive and repulsive characteristics of surface energies for fluids in microfluidic spaces; and the ability of inhomogeneous AC electrical fields to actuate droplet injection and the trapping, repulsion and transport of dielectric particles. These effects can be used to realize a programmable fluid processor (PFP) based on the dielectrophoretic (DEP) injection and manipulation of droplets within an immiscible carrier fluid over a reaction surface consisting of a Teflon-coated, addressable electrode array.

As used herein, “packet” and “particle” both refer to any compartmentalized matter. The terms may refer to a fluid packet or particle, an encapsulated packet or particle, and/or a solid packet or particle. A fluid packet or particle refers to one or more packets or particles of liquids or gases. A fluid packet or particle may refer to a droplet or bubble of a liquid or gas. A fluid packet or particle may refer to a droplet of water, a droplet of reagent, a droplet of solvent, a droplet of solution, a droplet of sample, a particle or cell suspension, a droplet of an intermediate product, a droplet of a final reaction product, or a droplet of any material. An example of a fluid packet or particle is a droplet of aqueous solution suspended in oil. The packet or particle may be encapsulated or a solid. Examples of solid packets or particles are a latex microsphere with reagent bound to its surface suspended in an aqueous solution, a cell, a spore, a granule of starch, dust, sediment and others. Methods for producing or obtaining packets or particle as defined herein are known in the art. Packets or particles may vary greatly in size and shape, as is known in the art. In exemplary embodiments described herein, packets or particles may have a diameter between about 100 nm and about 1 cm.

As used herein, an “array” refers to any grouping or arrangement. An array may be a linear arrangement of elements. It may also be a two dimensional grouping having columns and rows. Columns and rows need not be uniformly spaced or orthogonal. An array may also be any three dimensional arrangement.

As used herein the specification, “a” or “an” may mean one or more. As used herein in the claim(s), when used in conjunction with the word “comprising”, the words “a” or “an” may mean one or more than one. As used herein “another” may mean at least a second or more.

### VII. EXAMPLES

The following example is included to demonstrate a specific embodiment of the invention. It should be appreciated by those of skill in the art that the techniques disclosed in the example that follows represent techniques discovered by the inventors to function well in the practice of the invention, and thus can be considered to constitute specific, non-limiting modes for its practice. However, those of skill in the art should, in light of the present disclosure, appreciate that many changes can be made in the specific embodiment and still obtain a like or similar result without departing from the spirit and scope of the inventions defined by the claims.

### Example 1

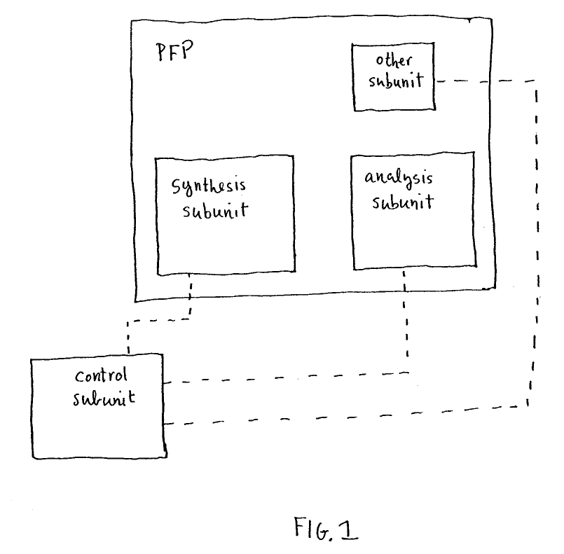

FIG. 1 shows a block diagram of a general embodiment of the present disclosure. There, a PFP device is illustrated, which includes various functional subunits, including an oligonucleotide synthesis subunit as described herein. Also shown is an analysis subunit and an “other” subunit, which may include any of the non-synthesis subunits described herein including but not limited to a subunit for lysing cells or for separating samples. Also shown is a control subunit, which may be coupled to the various other subunits as illustrated.

Having the benefit of the present disclosure, those having skill in the art will appreciate that any one or more of the subunits may be integral with or separate from an underlying device, which in this example is the PFP.

All of the techniques disclosed and claimed herein can be made and executed without undue experimentation in light of the present disclosure. While the compositions and methods of this invention have been described in terms of specific embodiments, it will be apparent to those of skill in the art that variations may be applied to the apparatuses and methods and in the steps or in the sequence of steps of the methods described herein without departing from the concept, spirit and scope of the invention.

### REFERENCES

Each of the following references is specifically incorporated herein by reference:

Ardrey, Electrospray Mass Spectrometry, Spectroscopy Europe, 4:10-18, 1992.

Axton et al., Journal of Medical Genetics April 1997;34(4):279-86.

Barany, F. Proc. Natl. Acad. Sci 88: 189-193, 1991.

Beaucage, and Lyer, Tetrahedron, 48:2223-2311, 1992.

Berkenkamp, Kirpekar, Hillenkamp, “Infrared MALDI mass spectrometry of large nucleic acids,” Science, 281:260-262, 1998.

Bonfield J K, Rada C, Staden R. Automated detection of point mutations using fluorescent sequence trace subtraction. Nucleic Acids Research 1998;26:3404-9.

Bruckner-Lea C J, Stottlemyre M S, Holman D A, Grate J W, Brockman F J, Chandler D P. “Rotating rod renewable microcolumns for automated, solid-phase DNA hybridization studies.” Anal Chem. September 1;72(17):4135-41, 2000.

Chandler D P, Holman D A, Brockman F J, Grate J W, and Bruckner-Lea C J, “Renewable Microcolumns for Solid Phase Nucleic Acid Separations and Analysis from Environmental Samples,” TrAC, Trends Anal. Chem., 9, 314-321 (2000).

Chandler et al., Anal. Chem., 2001, 73, 3784-3789.

Chow C S, Barton J K. “Recognition of G-U mismatches by tris(4,7-diphenyl-1,10-phenanthroline)rhodium(III).” Biochemistry Jun. 23, 1992;31(24):5423-9.

Cotton R G H. Current methods of mutation detection. Mutation Research 1993;285:125-44.

Cotton, Rodrigues, Campbell, “Reactivity of cytosine and thymine in single-base-pair mismatches with hydoxylamine and osmium tetroxide and its application to the study of mutations,” Proc. Natl. Acad. Sci. USA, 85(12):4397-4401, 1988.

Crain P F, McCloskey J A. “Applications of mass spectrometry to the characterization of oligonucleotides and nucleic acids.” Curr Opin Biotechnol. February 1998;9(1):25-34.

Deeble V J, Roberts E, Robinson M D, Woods C G, Bishop D T, Taylor G R. Comparison of Enzymne Mismatch Cleavage and Chemical Cleavage of Mismatch on a Defined Set of Heteroduplexes. Genetic Testing 1998 in press

Draghia et al., Human Mutation 1997;9(3):234-42.

Ekins R, Chu F W. “Microarrays: their origins and applications.” Trends Biotechnol. June 1999;17(6):217-8.

Ellis L A, Taylor G R, Banks R, Baumberg S. MutS binding protects heteroduplex DNA from exonuclease digestion in vitro—a simple method for detecting mutations. Nucleic Acids Research 1994;22:2710-1.

Ellis T P, Humphrey K E, Smith M J, Cotton R G., Hum Mutat 1998;11(5):345-53.

Fenn et al., J. Phys. Chem. 88, 4451-59, 1984.

Fishel R, Wilson T. MutS homologs in mammalian cells. Current Opinion in Genetics & Development 1997;7:105-13.

Freifelder, Physical Biochemistry Applications to Biochemistry and Molecular Biology, 2nd ed. Wm. Freeman and Co., New York, N.Y., 1982.

Frohman, In: PCR Protocols: A Guide To Methods And Applications, Academic Press, N.Y., 1990.

Gao, Yu, LeProust, Sonigo, Pellois, Zhang, “Oligonucleotide synthesis using solution photogenerated acids,” J. Am. Chem. Soc., 120:12698-12699, 1998.

Gascoyne, Wang, Huang, Becker, “Dielectrophoretic separation of cancer cells from blood,” IEEE Transactions on Industry Applications, 33(3):670-678, 1997.

Germain et al. Human Genetics December 1996; 98(6):719-26.

Gibbons R J, Bachoo S, Picketts D J, Higgs D R. Identification of mutations in ATRX by RNase mismatch cleavage. American Journal of Human Genetics 1997;61:1955-.

Giddings J C, Unified Separation Science, John Wiley & Sons, Inc, New York, N.Y. 1991.

Goldrick M M, Kimball G R, Liu Q, Martin L A, Sommer S S, Tseng J Y H. Nirca(Tm)—A Rapid Robust Method For Screening For Unknown Point Mutations.

Biotechniques 1996;21:106-12.

Gordon, L. K., and Haseltine, W. A.: Comparison of the cleavage of pyrimidine dimers by the bacteriophage T4 and Micrococcus luteus, Journ. Biol. Chem., 255, 12047 (1980).

Grange D K, Gottesman G S, Lewis M B, Marini J C. Detection Of Point Mutations In Type-I Collagen By RNase Digestion Of RNA RNA Hybrids. Nucleic Acids Research 1990;18:4227-36.

Hawkins G A, Hoffman L M. Accurate and efficient mutation detection and localization using base excision sequence scanning (BESS). American Journal of Human Genetics 1997;61:1279.

Hillenkamp et al. “Matrix Assisted UV-Laser Desorption/Ionization: A New Approach to Mass Spectrometry of Large Biomolecules,” in Biological Mass Spectrometry eds. Burlingame and McCloskey, Elsevier Science Publishers, Amsterdam, pp. 49-60, 1990.

Hsu I C, Yang Q P, Kahng M W, Xu J F. Detection of DNA point mutations with DNA mismatch repair enzymes. Carcinogenesis 1994;15:1657-62.

Ike, Y., Ikuta, S., Sato, M., Huang, T., & Itakura, K. (1983) “Solid phase synthesis of polynucleotides.” Nucl. Acid. Res., 11, 477.

Sambrook, E. F. Fritsch, and T. Maniatis, Molecular Cloning: A Laboratory Manual, 2 Ed., Cold spring Harbor Laboratory Press, Cold Spring Harbor, N.Y., 1989.

Jackson and Barton, “Recognition of DNA base mismatches by a rhodium intercalator,” J. Am. Chem. Soc., 119:12986-12987, 1997.

Jackson B A, Alekseyev V Y, Barton J K “A versatile mismatch recognition agent: specific cleavage of a plasmid DNA at a single base mispair.” Biochemistry Apr. 13, 1999;38(15):4655-62

Jackson, Alekseyev, Barton, “A versatile mismatch recognition agent: specific cleavage of a plasmid DNA at a single base mispair,” Biochemistry, 38(15):4655-4662, 1999.

Katzhendler, Cohen, Rahamim, Weisz, Ringel, Deutsch, “The effect of spacer, linkage and solid support on the synthesis of oligonucleotides,” Tetrahedron, 45:2777-2792, 1989.

Kisko J L, Barton J K., “Recognition of DNA base pair mismatches by a cyclometalated Rh(III) intercalator” Inorg Chem Oct. 16, 2000; 39(21):4942-9

Koster et al. Biomedical Environmental Mass Spectrometry 14: 111-116, 1987.

Laboratory Methods for the Detection of Mutations and Polymorphisms in DNA CRC Press 1997 Ed G R Taylor

Lahue R S, Au K G, Modrich P. DNA mismatch correction in a defined system. Science 1989;245:160-4.

Lambrinakos, Humphrey, babon, Ellis, Cotton, “Reactivity of potassium permanganate and tetraethylammonium chloride with mismatched bases and a simple mutation detection protocol,” Nuc. Acids Res., 27(8):1866-1874, 1999.

LeProust, Pellois, Yu, Zhang, Srivannavit, Gulari, Zhou, Gao, “Combinatorial screening method for synthesis optimization on a digital light-controlled microarray platform,” J. Comb. Chem., 2:355-360, 2000.

LeProust, Zhang, Yu, Gao, Xiang, Zhou, “Characterization of oligodeoxyribonucleotide synthesis on glass plates,” Nucleic Acids Res., 2001.

Lloyd, R. S., Hanawalt, P. C., and Dodson, M. L.: Processive action of T4 endonuclease V in UV-irradiated DNA, Nucleic Acids Res., 183, 109 (1980).

Lu A L, Hsu I C. Detection of single DNA-base mutations with mismatch repair enzymes. Genomics 1992;14:249-55.

Mashal R D, Koontz J, Sklar J. Detection of mutations by cleavage of DNA heteroduplexes with bacteriophage resolvases. Nature Genetics 1995;9:177-83.

Methods in Enzymology, Vol. 193: “Mass Spectrometry” (J. A. McCloskey, editor), 1990, Academic Press, New York.

Meyer and Day, BioTechniques 30:584-593, 2001.

Modrich P. Strand-specific mismatch repair in mammalian cells. Journal of Biological Chemistry 1997;272:24727-30.

Myers R M, Larin Z, Maniatis T. Detection Of Single Base Substitutions By Ribonuclease Cleavage At Mismatches In RNA-DNA Duplexes. Science 1985;230:1242-6.

Neddermann P, Gallinari P, Lettieri T, Schmid D, Truong O, Hsuan J J, Wiebauer K, Jiricny J. Cloning and expression of human G/T mismatch-specific thymine-DNA glycosylase. Journal of Biological Chemistry 1996;271:12767-74.

Oleykowski C A, Bronson-Mullins C R, Godwin A K, Yeung A T. Mutation detection using a novel plant endonuclease. Nucleic Acids Research 1988;16:4597-602.

Parsons B L, Heflich R H. Evaluation of MutS as a tool for direct measurement of point mutations in genomic DNA. Mutation Research-Fundemental and Molecular Mechanisms of Mutagenesis 1997;374:277-85.

Pellois, Wang, Gao, “Peptide synthesis based on t-Boc chemistry and solution photogenerated acids,” J. Comb. Chem., 2:349-354, 2000.

Pohler J G, Giraudpanis M E, Lilley D J. T4 endonuclease VII selects and alters the structure of the four-way DNA junction; Binding of a resolution-defective mutant enzyme. Journal of Molecular Biology 1996;260:678-96.

Roberts, Deeble, Woods, Taylor, “Potassium permanganate and tetraethylammonium chloride are a safe and effective substitute osmium tetroxide in solid-phase fluorescent chemical cleavage of mismatch,” Nuc. Acids Res., 25(16):3377-3378, 1997.

Rossiter B F, Caskey C T. Molecular scanning methods of mutation detection. Journal of Biological Chemistry 1990;265:12753-6.

Rowley G, Saad S, Gianelli F, Green P M. Ultrarapid mutation detection by multiplex, solid-phase chemical cleavage. Genomics 1995;30:574-82.

Saleeba, et al., Methods Enzymology, 217:286-295, 1993.

Schram, Methods Biochem Anal., 34: 203-287 1990.

Smith et al., Anal. Chem. 62, 882-89, 1990.

Smith J, Modrich P. Mutation detection with MutH, MutL, and MutS mismatch repair proteins. Proceedings Of The National Academy Of Sciences Of The United States Of America 1996;93:4374-9.

Smith J, Modrich P. Removal of polymerase-produced mutant sequences from PCR products. Proc Natl Acad Sci (US) 1997;94:6847-50. Smith et al., Mol. Gen. Genet., 224:447-481, 1990.

Stryer, L “Biochemistry”, Freeman and Co., 1995.

Taylor G R “Enzymatic and Chemical Cleavage Methods” http://www.leeds.ac.uk/cmgs/leedsdna/science/cleavage/review.htm

Taylor G R, Deeble J., “Enzymatic methods for mutation scanning.” Genet Anal February 1999;14(5-6):181-6.

Wang, Huang, Gascoyne, Becker, “Dielectrophoretic manipulation of particles,” IEEE Industrial App. Soc., 33(3):660-669, 1997.

Wang, Huang, Vykoukal, Becker, Gascoyne, “Cell separation by dielectrophoretic field-flow-fractionation,” Analytical Chemistry, 72(4):832-839, 2000.

Wang, Pethig, Jones, “Relationship of dielectrophoretic and electrorotational behavior exhibited by polarized particles,” J. Phys. D Appl. Phys., 25:905-912, 1992.

Wang, Vykoukal, Becker, Gascoyne, “Separation of polystyrene microbeads using dielectrophoretic/gravitational field-flow-fractionation,” Biophys. J., 12:3846, 1998.

Wasserman, Tao, Whitesides, “Structure and reactivity of alkylsiloxane monolayers formed by reaction of alkytrichlorosilanes on silicon substrates,” Langmuir, 5:1074-1087, 1989.

White, Giraud-Panis, Pohler, Lilley, “Recognition and manipulation of branched DNA structure by junction-resolving enzymes,” J. Mol. Biol., 269:647-664, 1997.

Williams et al., Science, 246: 1585-87, 1989

Wu, D. Y. and R. B. Wallace Gene, 76:245-254, 1989.

Yao and Kow, “Further characterization of E. coli endonuclease V″ mechanism of recognition for deoxyinosine, deoxyuridine, and base mismatches in DNA,” J. Biol. Chem., 272(49):30774-30779, 1997.

Youil et al., Hum Mutat July 2000;16(1):92-3.

Youil R, Kemper B, Cotton R G H. Detection of 81 of 81 known mouse beta-globin promoter mutations with T4 Endonuclease-VII—The EMC Method. Genomics 1996;32:431-5.

Youil, Kemper, Cotton, “Screening for mutations by enzyme mismatch cleavage with T4 endonuclease VII,” Proc. Natl. Acad. Sci. USA, 92(1):87-91, 1995.

What is claimed is:

1. A microfluidic device comprising:
an oligonucleotide synthesis subunit comprising a reaction surface and means for generating a manipulation force and routing for packet delivery;
an analysis or diagnostic subunit; and
a control subunit comprising a program to direct oligonucleotide synthesis.

2. The device of claim 1, comprising a programmable fluidic processor (PFP).

3. The device of claim 2, wherein a sequence to be synthesized is downloaded to the control electronics of said PFP.

4. The device of claim 3, wherein sequences are downloaded to the control electronics of said PFP.

5. The device of claim 2, wherein said PFP is configured to act as a program manifold.

6. The device of claim 5, wherein said program manifold routes and delivers one or more reagents.

7. The device of claim 1, wherein said synthesis subunit comprises wall-less channels.

8. The device of claim 1, wherein said synthesis subunit is adapted for use with phosphoramidite chemistry.

9. The device of claim 8, wherein said phosphoramidite chemistry is modified phosphoramidite chemistry.

10. The device of claim 1, wherein said synthesis subunit comprises a bead or reagent reservoir.

11. The device of claim 1, wherein said routing for packet delivery comprises using dielectrophoresis.

12. The device of claim 1,wherein said analysis or diagnostic subunit comprises wall-less channels.

13. The device of claim 1, wherein said analysis or diagnostic subunit is adapted for measuring impedance, conductance, electrophoretic movements, fluorescence, mass ion peaks, hybridization, ligase, base addition, and/or fluocytometry.

14. A method of using the device of claim 1.

15. A method for analyzing a sample comprising:
synthesizing an oligonucleotide in a solid-phase oligonucleotide synthesis subunit, wherein said subunit comprises a reaction surface and means for generating a manipulation force and routing for packet delivery;
controlling said synthesis with a control subunit wherein said control subunit is programmed for the automatic synthesis of oligonucleotides; and
analyzing said sample in an analysis or diagnostic subunit.

16. The method of claim 15, wherein said synthesis subunit or said analysis or diagnostic subunit comprises a programmable fluidic processor (PFP).

17. The method of claim 16, further comprising downloading the sequence of said oligonucleotide to the control electronics of the PFP.

18. The method of claim 17, further comprising downloading 2-1000 oligonucleotide sequences to the control electronics of said PFP.

19. The method of claim 16, wherein said PFP is configured to act as a program manifold.

20. The method of claim 19, wherein said program manifold is used for reagent routing and delivery.

21. The method of claim 15, wherein said analysis is controlled by said control subunit.

22. The method of claim 15, wherein said oligonucleotide is 10-20 base pairs in length.

23. The method of claim 15, wherein synthesizing comprises using phosphoramidite chemistry.

24. The device of claim 23, wherein said phosphoramidite chemistry is modified phosphoramidite chemistry.

25. The method of claim 15, further comprising using a protecting group during oligonucleotide synthesis.

26. The method of claim 25, wherein said protecting group comprises a sugar and a phosphate.

27. The method of claim 25, wherein said protecting group is removed by chemical or photochemical means.

28. The method of claim 27, further comprising using laser assisted deprotection.

29. The method of claim 15, further comprising analysis of said sample with MALDI-TOF MS.

30. The method of claim 15, further comprising synthesizing a second oligonucleotide, wherein the synthesis of two oligonucleotides occur in parallel.

31. The method of claim 15, further comprising synthesizing a second oligonucleotide, wherein the synthesis of two oligonucleotides occur sequentially.

32. The method of claim 15, wherein said solid-phase comprises dielectrically engineered beads.

33. The method of claim 32, wherein said beads are 2-50 μm in diameter.

34. The method of claim 32, wherein said beads are manipulated by dielectrophoresis.

35. The method of claim 32, wherein said beads are gold coated polystyrene beads.

36. The method of claim 32, wherein said beads are coated with a phospholipid.

37. The method of claim 32, wherein said beads are coated with a polyethylene glycol.

38. The method of claim 15, wherein said analyzing comprises gene discovery, SNP analysis, disease diagnosis, drug discovery, toxicological research, detection of chemical and biological warfare agents, analysis of terrorism agents, pathogen detection, pollution monitoring, water monitoring, fertilizer analysis, food pathogen detection, quality control and blending, massively parallel molecular biological protocols, genetic engineering, oncogene detection, or pharmaceutical development and testing.

39. The method of claim 15, wherein analyzing said sample comprises determining the interaction of said sample with said oligonucleotide.

40. The method of claim 15, wherein said oligonucleotide is synthesized immediately before analysis.

41. The method of claim 15, further comprising proofreading said oligonucleotide.

42. The method of claim 15, further comprising analyzing said sample a second time.

43. The method of claim 42, wherein said second analysis occurs in an analysis or diagnostic subunit.

44. The method of claim 42, wherein said second analysis occurs after removing said sample from said analysis or diagnostic subunit.

45. The method of claim 15, further comprising analyzing a second sample.

46. The method of claim 45, wherein synthesizing an oligonucleotide in a solid-phase oligonucleotide synthesis subunit for analysis of said second sample occurs before said analyzing the first sample in an analysis or diagnostic subunit is completed.

47. The method of claim 45, wherein said second sample is analyzed simultaneously with the first sample.

48. An apparatus for performing the method of claim 15.
